(19) United States
(12) Patent Application Publication
(10) Pub. No.: US 2002/0137161 A1
(43) Pub. Date: Sep. 26, 2002

(54) Protein expression and structure solution using specific fusion vectors

(75) Inventors: Dietmar J. Manstein, Heidelberg (DE); Jon F. Kull, Enfield, NH (US); Menno L. W. Knetsch, Maastricht (NL); Hartmut H. Niemann, Heidelberg (DE)

Correspondence Address: Candice J. Clement, Esq., HESLIN ROTHENBERG FARLEY & MESITI, P.C., 5 Columbia Circle, Albany, NY, 12203, US

(73) Assignee: Max-Planck-Gesellschaft zur Forderung der Wissenschaftern e.V., Munchen (DE)

(21) Appl. No.: 10/044,303

(22) Filed: Jan. 11, 2002

(30) Foreign Application Priority Data
Jan. 12, 2001 (EP) 01100762.2

(51) Int. Cl. C12N 9/00; C07H 21/04; C12P 21/02; C12N 5/06

(52) U.S. Cl. 435/183; 435/69.1; 435/325; 435/320.1; 536/23.2

(57) ABSTRACT

A recombinant protein comprised of an amino acid sequence of a motor protein, a target protein of interest, and optionally, a linker sequence between the two proteins, is disclosed. Also disclosed are a DNA sequence encoding such a recombinant protein, a vector expressing such a recombinant protein, a host cell transformed with such a vector, a method for producing such a recombinant protein, and methods for purification, crystallization and structure elucidation of such a recombinant protein. In preferred embodiments of the invention, the motor protein is a member of the kinesin or myosin superfamilies, or an analog, fragment or derivative thereof.

### CLAIM FOR FOREIGN PRIORITY

This application claims priority from European Patent Application 01100762.2, filed Jan. 12, 2001. The entire contents of the prior application is incorporated herein by reference.

### Reference to Sequence Listing

This application includes a “Sequence Listing” provided in computer readable form, “PatentIn Ver. 2.1,” the entire contents of which is incorporated herein by reference. A paper copy of the “Sequence Listing” is also provided.

### BACKGROUND OF THE INVENTION

The present invention relates to a recombinant protein comprised of an amino acid sequence of a motor protein, a target protein of interest, and optionally, a linker sequence between the two proteins. The invention also relates to a DNA sequence encoding such a recombinant protein, a vector expressing such a recombinant protein, a host cell transformed with such a vector, a method for producing such a recombinant protein, and methods for purification, crystallization and structure elucidation of such a recombinant protein.

The first step, and perhaps the single most important step, in the crystallization of a macromolecule, e.g., a protein, is its purification. Any impurities of the protein solution to be used for crystallization may impair crystal quality or, even worse, preclude the formation of crystals at all.

Procedures for accomplishing the highest degree of purification possible have been under development for more than 200 years, and recent times have seen an explosion in the invention of new methods and refinement of old. There is a variety of methods that exemplify how problems of protein purification for protein analysis or protein crystallization have been approached.

One such method is fractionation with salts and other precipitants. Hereby, proteins are precipitated from a complex mixture (e.g., a physiological fluid) by addition of various concentrations of different salts. Because individual proteins precipitate at different salt concentrations, this “salting” out phenomenon provided a method for selectively precipitating, and thereby purifying, unique proteins from a mixture (Morris and Morris, “Separation methods in biochemistry,” Pitman, London, GB, 1964). A minor disadvantage of salt fractionation is that protein preparations, be they supernatants or precipitates, are left with high residuals of salt. This may seriously interfere with the evaluation of activity, purity and with subsequent purification procedures. The most common of these methods is dialysis in celluloid or collodion tubes.

Apart from varying the concentration of a salt, proteins may be selectively precipitated and fractionated by the addition of a variety of organic solvents (Cohn et al., “Crystallization of serum albumin from ethanol/water mixtures”, J. Am. Chem. Soc., 69:1753, 1947). This is generally carried out at sub-zero temperatures ranging to −30° C. to enhance the precipitation effect and to minimize the denaturation of the protein. In addition to salt and organic solvents, other materials have been used to precipitate and fractionate a mixture of proteins. Some of these materials are, for example, protamine (a mixture of small basic proteins) and polyeneimine (a basic organic polymer), which apparently cross-links some protein via electrostatic bridges. Moreover, metal ions or organic polymers, such as polyethylene glycol (PEG), were extensively used for purification purposes. PEG seems to act as a hybrid between an alcohol and a salt and their precise properties may vary as a function of mean polymer length.

Still another method of protein purification is the selection of proteins with heat or pH. pH is effective because most proteins exhibit pH-dependent solubility minima and precipitate or even crystallize from solution at particular values, whereas the property of protein heat stability may sometimes provide a valuable purification step.

Other protein purification methods based on physical techniques are also well-known to a person skilled in the art. Centrifugation, for example, has to be mentioned, whereby a solution containing multiple components varying in weight, size, and density is deployed in a tube and rotated at high angular velocity. An almost preparative centrifugation is conducted on some gradient with various density from the top to the bottom of the centrifuge tube. Two common techniques utilized in connection with density gradient separation are sedimentation velocity and sedimentation equilibrium or isocyanic centrifugation. Furthermore, electrophoretic separation methods (Svensson, “Preparative electrophoresis and ionophoresis,” Adv. Protein Chem., 4:251, 1947) are routinely used and are based on the application of an electrical field across and insoluble, porous support medium permeated by a buffer solution. Dependent on the net charge of the proteins to be separated they will experience and electromotive force and migrate toward one electrode (cathode or anode). For the separation of proteins, polyacrylamide gels as support medium have shown to have almost ideal properties.

Finally, chromatographic methods are especially well suited to separate proteins and to purify the target protein for later crystallization steps. Classic ion exchange chromatography is simply conducted by packing a vertical hollow glass column with an insoluble resin or colloidal matrix that exhibits an array of positively charged (anion exchange chromatography) or negatively charged chemical groups (ration exchange chromatography). Ion exchange chromatography is based on the fact that a positively charged protein will be retarded or bound to electrostatic interactions with a matrix carrying negatively charged groups or vice-versa for negatively charged proteins. Dependent on their respective net charge the proteins to be separated will appear in the eluent sequentially with time (or volume). Molecules tightly bound to the matrix may be eluted from the column by competition with other charged ions. In contrast to ion exchange chromatography, molecular sieve chromatography (also called gel permeation chromatography) separates molecules on the basis of molecular weight and shape. Hereby, macromolecules, like proteins, are induced to flow by gravity or pressure through a column containing a matrix of microscopic beads perforated with a vast network of channels. Thereby, the high molecular sieving effect will influence the speed in passing from the top to the bottom of the column leading to the inverse effect that larger molecules will appear first in the column eluent. Finally, absorption chromatography, HPLC (high performance liquid chromatography), and affinity chromatography are also well established as biochemical purification methods.

All of the above-mentioned methods exhibit certain advantages and disadvantages. Consequently, the person skilled in the art will choose the purification method which appears to be most appropriate for a given system.

In recent years many purification methods have begun to take advantage of recombinant proteins (Kane and Hartley, “Development of expression systems for production of high levels of protein,” Trends Biotechnol., 6:95, 1988). Recombinant proteins are produced by recombinant DNA techniques in bacteria, yeast or other organisms such as virus infected mammalian or insect cells. The advantage of recombinant proteins is based on genetically designed elements, that aid the biochemist in applying one of the aforementioned physical or biochemical purification methods. For example, a series of histidine residues, or a “His-tag”, may be appended to the carboxyl terminus of a recombinant protein. Such a histidine appendix makes it easier to isolate the expressed protein on a copper or nickel containing chromatographic resin, the latter being available commercially in prepacked columns.

A second procedure in wide use for the purification of recombinant proteins is the fusion of an expressed protein with the enzyme glutathione sulfur transferase (GST). This enzyme has a very high affinity for the small peptide glutathione. Following expression of the protein, an extract of the cells is passed over a small chromatography column containing a matrix conjugated with glutathione. The chimeric protein is then reversibly bound on the column through the GST, contaminants are washed from the column, and finally the recombinant protein is eluted with free glutathione and collected. The GST may then be cleaved from the chimer by a specific protease to produce the free recombinant protein. Again, the chromatographic matrix may be obtained commercially in prepacked columns.

Furthermore, e.g., pMAL™ (by New England Biolabs Inc.) is used as protein fusion and purification system prior art. This system comprises the insertion of the cloned gene into a pMAL™ vector downstream from the malE gene, which encodes maltose binding protein (MBP). The fusion protein (target protein and MBP) is expressed in large quantities and purified by affinity chromatography for MBP using amylose resin. Finally, MBP is cleaved from the target protein by a specific protease.

These techniques utilizing recombinant proteins allow one to obtain extraordinarily pure fractions of the target protein. However, advantageous conditions for purification, crystallization and structural analysis have to be tested using the MBP/target protein or GST/target protein fusion systems for each single recombinant and/or target protein. In particular, there are still complex chromatographic (in vitro) purification steps required for obtaining pure fractions of the target protein and further steps of analysis, like crystallization or structure determination, are complicated by the unknown properties of the target protein, such as, for example, the crystallization conditions of a specific target protein purified as MBP or GST fusion protein.

### SUMMARY OF THE INVENTION

The object of the present invention is to overcome the above-mentioned disadvantages of the prior art, and particularly to provide a system that considerably reduces the time effort for purification and subsequent crystallization as well as structure determination of any protein to be analyzed.

The principles of the present invention provide a recombinant protein comprised of an amino acid sequence of a motor protein, a target protein of interest, and optionally, a linker sequence between the two proteins. The invention also relates to a DNA sequence encoding such a recombinant protein, a vector expressing such a recombinant protein, a host cell transformed with such a vector, a method for producing such a recombinant protein, and methods for purification, crystallization and structure elucidation of such a recombinant protein.

In one aspect, the present invention provides recombinant proteins comprising:

(1) an amino acid sequence of a member of the myosin or kinesin protein superfamilies or an amino acid sequence of an analog, fragment or derivative of a member of the myosin or kinesin protein superfamilies;

(2) any amino acid sequence of at least 20 amino acids in length (target protein sequence); and optionally,

(3) a linker region of at least 2 amino acids between components (1) and (2).

It is within the scope of this invention that components (1) and (2) are directly fused together without insertion of linker sequence (3).

In accordance with the principles of the invention, component (1) may comprise any protein or fragment, derivative or analog thereof, which binds to any molecule or structure of the cytoskeleton or a cell membrane in a ligand dependent manner. Particularly preferred are molecules, which exhibit a flexible region, particularly at the molecules C-terminal region, in order to sample for multiple conformations.

Component (1) may also comprise an amino acid sequence of an analog, fragment or derivative of a member of the myosin or kinesin protein superfamilies. The preparation of such analogs, fragments and derivatives is by a standard procedure (Sambrook et al., “Molecular Cloning: A Laboratory Manual,” Cold Spring Harbor, N.Y., 1989) in which in the DNA sequences encoding the inventive recombinant protein, one or more codons may be deleted, added or substituted by another, to yield analogs having at least one amino acid residue change with respect to the native recombinant protein, particularly with respect to the native amino acid sequence of component (1) or (2) of the recombinant protein of the invention.

Analogs that substantially correspond to the native sequence of one or more components of the inventive recombinant protein are those polypeptides, in which one or more amino acids of the native protein's amino acid sequence has/have been replaced by another amino acid, deleted and/or inserted.

In a preferred embodiment of the present invention, the resulting components ((1) or (2)) being incorporated into the recombinant protein of the invention exhibit substantially the same or even higher biological activity as the corresponding native protein to which it corresponds or exhibit at least structurally similar properties as the native protein to which the component corresponds. In order to substantially correspond to the native sequence of component (1) or (2) of the recombinant protein of the invention, the changes in the sequence of the components are generally and preferably relatively minor, such as isoforms. Although the number of changes may be more than 10, preferably there are no more than 10 changes, more preferable no more than 5 and most preferably no more than 3 changes in component (1) or (2) as compared to the respective native sequence.

While any technique may be used to find potentially biologically active sequences of a component of the inventive recombinant protein, which substantially correspond to the respective native proteins, one such technique is the use of conventional mutagenesis techniques on the DNA encoding the protein, resulting in a few modifications. The sequences used for component (1) or (2) in the recombinant protein of the invention which are expressed by such clones, may then be screened for their ability e.g., to bind to their native binding partners, mediate activity etc., in other words fulfil their biological role.

Conservative “changes” are those changes which would not be expected to change the activity of the protein and are usually the first to be screened as these would not be expected to substantially change size, charge or structure of the polypeptide sequence used as component in the recombinant protein of the invention and thus would not be expected to change the biological properties of the corresponding native sequence. For example, conservative substitutions are assumed, if: (a) small aliphatic, non-polar or slightly polar residues are substituted by other residues belonging to the same group; (b) polar negatively charged residues and their amides are exchanged for other residues belonging to the same group; (c) polar positively charged residues are exchanged for polar positively residues; (d) large aliphatic non-polar residues are exchanged for large aliphatic non-polar residues; or (e) finally, aromatic residues are substituted by other aromatic residues.

In most cases, in the context of the present invention, analogs being used as component (1) or (2) of the recombinant protein of the invention are defined as sequences with substitutions which do not produce radical changes in the characteristics of the corresponding native protein or polypeptide molecule. Characteristics may be the specific secondary structure of a sequence, e.g., α-helix or β-sheet, as well as its specific biological activity.

It is noted that apart from sequences being used as component (1) or (2) for a recombinant protein according to the present invention, which are based on conservative substitutions as discussed above, analogs with more random changes, which lead to a radical or more radical change in biological activity or structure of the analog as compared to the native sequence are also within the scope of the present invention.

At the genetic level, these analogs are generally prepared by site-directed mutagenesis of nucleotides in the DNA encoding the inventive recombinant protein or the component of the recombinant protein, respectively, thereby producing DNA encoding the analog and thereafter synthesizing the DNA and expressing the polypeptide in recombinant cell culture. Reference is made to Ausübel et al., “Current Protocols in Molecular Biology,” Green Publications and Wiley Intersigns, New York, N.Y., 1987-1995; and Sambrook et al., “Molecular Cloning: Laboratory Manual,” Cold Spring Harbor Laboratory, New York, 1989, the entire disclosures of which are incorporated herein by reference.

Furthermore, site-specific mutagenesis allows the production of analogs through the use of specific oligonucleotide sequences that encode the DNA sequence of the desired mutation. The technique of site-directed mutagenesis is exemplified by publications such as Adelman et al., DNA, 2:183 (1983), the entire disclosure of which is incorporated herein by reference. Typical vectors useful in site-directed mutagenesis include vectors such as M13-phage, for example as disclosed by Messing et al., “3rd Cleveland Symposium on Macromolecules and recombinant DNA,” editor A. Walton, Elsevier, Amsterdam (1981), the entire disclosure of which is incorporated herein by reference.

As far as derivatives of the native sequence of components of the recombinant protein of the present invention are concerned, derivatives may be prepared by standard modifications of the side groups of one or more amino acid residues of the recombinant protein of the invention, its analogs or fragments or by conjugation of the native sequence used as component (1) or (2) of the inventive recombinant protein, its analogs or fragments, to another molecule, e.g., an antibody, enzyme, receptor, etc.

Accordingly, “derivatives” as used herein cover derivatives that may be prepared from the functional groups occurring as side chains on the residues or from the N- or C-terminal groups by means known in the art. Derivatives may have chemical moieties such as carbohydrates or phosphate residues. For example, derivatives may include aliphatic esters of the carboxyl groups, amides of the carboxyl group by reaction with ammonia or with primary or secondary amines, N-acyl derivatives or free amino groups of the amino acid residues formed with acyl moieties or O-acyl derivatives of free hydroxyl groups (for example of seryl or threonyl residues) formed with acyl moieties. The term derivative will also include, all polypeptide sequences for a particular component ((1) and/or (2)) of the recombinant protein sequence which are larger in sequence than the corresponding native sequence. The addition of at least one, typically more than 10 amino acids may take place intrasequentially or at the N- or C-terminus of the sequence of component (1) and/or (2) of an inventive recombinant protein. In a preferred embodiment of the present invention, additional amino acids are appended to the N-terminus of component (1) or the C-terminus of component (2) coinciding with the N-terminus and the C-terminus of the inventive recombinant protein.

In another preferred embodiment, additional amino acid sequences are inserted intrasequentially, preferably in such a way that the secondary and/or tertiary structure is not destroyed. Typically these insertions are placed at the surface of the protein, e.g., in β-bends. Preferably, one or more S-containing residues (particularly Cys) are inserted or other residues with a potential for binding heavy metal atoms (e.g., Hg-ions). The introduction of additional heavy metal binding residues at sites on the surface of the recombinant protein of the invention may be by substitution and/or deletion of native binding residues in order to create novel heavy metal atom binding sites. Such a procedure is particularly suitable for gaining additional phasing information for structure determination of large protein complexes by X-ray crystallography.

In a non-limiting manner, “tag”-sequences may be contained in the recombinant protein and, particularly, may be added to the N- or C-terminus of the recombinant protein of the invention. These “tag”-sequences typically have antigenic character for commercially available antibodies, e.g., an N-terminal “Flag-tag” having the sequence DYKDDDDK (one-letter-code). Other suitable “tag”-sequences are, for example, N- or C-terminal polyhistidine tags.

Furthermore, component (1) and/or (2) as parts of the recombinant protein of the invention may be fusion proteins. Particularly preferred are sequences fused to the N-terminus of the native sequence of component (1) or to the N-terminus of an analog, derivative or fragment thereof. For example, component (1) of the recombinant protein may be fused N-terminally to a marker protein, e.g., an enzyme marker or a fluorescence marker, such as GFP (green fluorescence protein), or any sequence being suitable as epitope for an antibody or even to an antibody or an antibody fragment itself.

Finally, “fragments” of the native sequence of any protein being used as component (1) or (2) of the recombinant protein according to the present invention may be used, e.g., fragments of proteins of the myosin or kinesin protein superfamilies, particularly fragments being deleted C-terminally, the deletion comprising at least ten, and more preferably at least 50 amino acids. However, the fragment of the native sequence may also contain deletions at the N- and/or the C-terminus and/or intrasequentially in component (1) and/or component (2) of a recombinant protein of the invention.

In a preferred embodiment, component (1) consists of a fragment comprising the catalytic domain of a member of the myosin or kinesin protein superfamilies of any eukaryotic organism. In other words, component (1) corresponds preferably to a fragment containing the myosin or kinesin motor domain. Within the scope of the present invention are therefore recombinant proteins characterized in that they contain as component (1) an amino acid sequence for the motor domain of a kinesin or myosin family member or an analog, fragment or derivative thereof.

In a preferred embodiment of the present invention, the recombinant protein according to the present invention contains as component (1) an amino acid sequence of a member of the myosin I, II, III, IV, V, VI, VIII, X, or XI or a member of kinesin I or II families or an amino acid sequence of an analog, fragment or derivative of a member of the aforementioned myosin and kinesin families. Preferably, component (1) contains a member of the myosin II family of any eukaryotic organism or an analog, fragment or derivative thereof.

Still further preferred, component (1) contains myosin II of Dictyostelium or an analog, fragment or derivative thereof. Further preferred embodiments of the present invention for component (1) are proteins containing the motor domains of smooth muscle myosin II (e.g., chicken gizzard myosin), vertebrate or amoeboid forms of myosin I (bovine brushborder myosin), Dictyostelium myoID, vertebrate myosin V, myosin VI, Toxoplasma gondii (e.g., TgMyoA) and Plasmodium sp. myosin XIV, vertebrate kinesin (human kinesin I), amoeboid or fungal kinesins (e.g., Dictyostelium kinesin 7).

Preferably, a recombinant protein according to the present invention contains as linker component (3) a stretch of at least 3 amino acids, more preferably 5 amino acids, and still further preferably, 10 amino acids. Particularly preferred is a linker sequence which contains a protease cleavage site. A recognition sequence for any protease may be used, for example, the cleavage site may contain the recognition sequence for factor Xa, thrombin or for the protease TEV (recognition sequence: ENLYFQG) or the Soldati protease. However, as discussed previously, linker component (3) is optional, and it is within the scope of this invention that components (1) and (2) are directly fused together without insertion of a linker sequence.

If linker component (3) consists of three amino acids, it is preferred to chose a sequence with at least one Gly residue, particularly in the second position of the linker stretch. More preferred, however, is a linker with the sequence: N-Leu-Gly-Arg-C or N-Leu-Gly-Ser-C.

As component (2) (the target protein), preferred recombinant proteins of the present invention may contain the sequence of an esterase, hydrolase, phosphatase, kinase, protease, channel, structural protein (e.g., coronin, spectrin), receptor, particularly a neuronal or immunologically relevant receptor (e.g., superfamily of TNF receptors), transcription factor, DNA/RNA-binding protein, lipoprotein, glycoprotein or an analog, derivative or fragment thereof.

A recombinant protein according to the present invention may have as component (1) an amino acid sequence as exhibited in FIG. 6 (SEQ ID NO. 1) or an analog, derivative and/or fragment thereof. It is preferred to combine the sequence of FIG. 6 with a linker sequence (3) containing a protease recognition site as exemplified above or the amino acid sequence Leu-Gly-Ser. Still further preferred is a recombinant protein having a sequence as shown in FIG. 7 (SEQ ID NO. 2).

A second aspect of the present invention relates to a DNA sequence which contains a sequence which codes for an amino acid sequence of a recombinant protein according to the present invention. In particular, the present invention provides a DNA sequence selected from the group consisting of:

(a) a cDNA sequence derived from the coding region of a recombinant protein according to the present invention;

(b) DNA sequences capable of hybridization to a sequence of (a) under moderately stringent conditions; and

(c) DNA sequences which are degenerate as a result of the genetic code to the DNA sequences defined in (a) and (b), above.

Another specific embodiment of the above DNA sequence of the invention is a DNA sequence comprising at least part of a sequence encoding for a recombinant protein as depicted in FIG. 8 (SEQ ID NO. 3) particularly the segment of FIG. 8 which codes for the myosin motor domain. Nucleic acid stretches encoding for a recombinant protein of the present invention may be detected, obtained and/or modified, in vitro, in-situ and/or in vivo, by the use of known DNA or RNA amplification techniques, such as polymerase chain reaction (PCR) and chemical oligonucleotide synthesis.

PCR allows for the amplification (increase in number) of a specific DNA sequence by repeated DNA polymerase reactions. This reaction may be used as a replacement for cloning. All that is required is a knowledge of the nucleic acid sequence. In order to carry out PCR, primers are designed which are complementary to the sequence of interest. The primers are then generated by automated DNA synthesis. Because primers may be defined to hybridize to any part of the gene, conditions may be created such that mismatches in the complementary base pairing may be tolerated. Amplification of these mismatch regions may lead to the synthesis of a mutagenized product resulting in the generation of a polypeptide with new properties (site-directed mutagenesis).

By coupling complementary DNA (cDNA) synthesis, using reverse transcriptase, with PCR, RNA may be used as the starting material for the synthesis of a recombinant protein of the invention. Furthermore, PCR primers may be designed to incorporate new restriction sites or other features such as termination codons at the end of the segment to be amplified. This placement of restriction sites at the 5′ and 3′ ends of the amplified nucleic sequence allows for a gene sequence including a recombinant protein of the invention or a fragment thereof to be custom designed for ligation with other sequences and/or cloning sites in vectors.

PCR and other methods of amplification of RNA and/or DNA are well known in the art and may be used according to the present invention without undue experimentation. Known methods of DNA and RNA amplification include PCR and related amplification processes (Innes et al., PCR Protocols: A Guide to Method and Amplification) and RNA mediated amplification which uses antisense RNA to the target sequence as a template for double stranded DNA synthesis (see, e.g., U.S. Pat. No. 5,130,238, the entirety of which is incorporated herein by reference).

In an analogous fashion, a recombinant protein of the invention being composed of components 1, (2) and (3) as defined above may be prepared, whereby components (1), (2) and (3) are ligated on a genetic level forming a DNA sequence of the invention, which is used to express a recombinant protein of the invention in a suitable host system.

Also provided by the present invention are vectors encoding the above recombinant protein, and analogs, fragments or derivatives of the invention, which contain the above DNA sequence of the invention. Such vectors are capable of being expressed in suitable eukaryotic or prokaryotic host cells. Particularly preferred are vectors of the invention, which are capable of being expressed in cells of the species Dictyostelium.

In an expression vector of the present, invention the DNA sequence is operably linked to a promoter, preferably linked upstream. The promoter will preferably be an eukaryotic promoter, particularly a constitutive promoter. The transcription of a DNA sequence according to the invention in cells of higher eukaryotes may be derived from viral genomes. Examples would be polyoma viruses, retroviruses, adenoviruses, cytomegaloviruses, SV40 and the like. With mammalian cells, a possibility would be the β-actin promoter. In the current invention, the actin15 promoter is particularly preferred for expression in Dictyostelium.

If appropriate, other regulating elements of transcription and/or translation will be provided. Particularly preferred are cis-acting elements, such as enhancer sequences, which usually include 10 to 300 base pairs and act upon the promoter to raise the transcription rate. These may be arranged in the 3′ or 5′ position of the DNA sequence according to the invention, in the coding sequence itself, or in an intron sequence which is cut out by splice procedures. Further regulating elements may serve to regulate transcription termination, so that the expression of mRNA is involved.

If necessary, the expression vector with the DNA of the invention are developed as shuttle vectors, that is, they are able to replicate in a host system and can then be transfected into another host system for purposes of expression. For instance, a vector might first be cloned in E. coli and then be inoculated into Dictyostelium, yeast or any mammalian cell for expression.

Typically, such expression and cloning vectors include at least one selection gene exercising a marker function. A selection gene allows host cells to survive or grow after being transformed by the vector. Typical selection genes code for proteins that permit resistance toward antibiotics or other toxins. This, for instance, includes puromycin, ampicillin or neomycin.

The principles of the present invention also provide host cells, and particularly eukaryotic host cells, transformed with an expression vector according to the invention. Appropriate host cells for cloning or expressing the DNA sequences are prokaryotic cells, yeast or higher eukaryotic cells. In a preferred embodiment, cells for expressing DNA sequences according to the invention are selected from multicellular organisms. This also takes place before the background of the function of component (1) of the recombinant protein of the invention to elements of the cytoskeleton (e.g., actin, microtubules or components of the cell membrane or membrane of any intracellular organelle, e.g., mitochondria). In principle any eukaryotic cell may be used as host cell, although cells of mammals such as monkeys, mice, rats, hamster or humans, are preferred. Particularly preferred are cells from the species Dictyostelium.

The present invention relates in a further aspect to a method for producing a recombinant protein according to the invention, the method comprises the following steps:

(a) preparing a vector according to the invention;

(b) transforming eukaryotic host cells with a vector obtainable from step (a); and

(c) growing transformed host cells of the invention and obtainable from step (b) under conditions suitable for the expression of the recombinant protein.

The expression method of the invention allows for overexpression of any target protein or polypeptide of at least 20 amino acid length (component (2)), as a segment of the recombinant protein of the invention. Accordingly, huge amounts of target protein as part of a recombinant protein of the invention are produced by the method of the invention. It is preferred within the scope of the present invention to concentrate the overexpressed recombinant protein in the cell. This is achieved by constructing recombinant proteins of the invention, which do not carry any leader sequences for secretion out of the transformed host cell.

Another aspect of the present invention is a method for purifying a recombinant protein of the invention or any other recombinant protein containing an amino acid sequence binding to cytoskeleton (actin or microtubules or proteins being bound to actin in the cell) or membrane (e.g., inner cell membrane or outer or inner membrane of a cell organelle) structures and another amino acid sequence (the target sequence to be analyzed), the method comprises:

(a) preparing a vector according to the invention or a vector encoding for any recombinant protein (as disclosed above);

(b) transforming eukaryotic host cells with a vector obtainable from step (a);

(c) growing transformed host cells according to the invention and/or obtainable from step (b) under conditions suitable for the overexpression of the recombinant protein;

(d) purifying overexpressed recombinant protein by binding to endogenous elements or structures of the cytoskeleton or membrane, such as actin or microtubules, of the eukaryotic host cell; and

(e) releasing bound recombinant protein from these structures or elements, preferably actin or microtubules.

In a preferred embodiment of this method, step (e), the releasing step, involves a separation from the structures or elements of the cell by adding a substrate, be it a natural or non-natural substrate, of component (1) of the recombinant protein. Whereas in general the natural substrate will be used, it may be preferable in certain cases to use a non-natural substrate of component (1), such as, for example, GTP or (nucleotide) analogues (where ATP is the natural substrate), for releasing purposes. In general, any substrate with the potential to release the bound recombinant protein, particularly by binding to the component (1) of the recombinant protein from the cell structure or element is suitable to be used for step (e). It will be appreciated that a method of the invention using a member of the kinesin or myosin superfamily or a derivative, fragment or analog thereof as component (1) is particularly preferred, if it is characterized in the addition of ATP, which is the natural substrate for these proteins with motility function.

In yet a further preferred embodiment, the purification method of the invention comprises an additional step (f). Step (f) may typically provide at least one additional in vitro purification step, whereby all common purification procedures available may be provided, for instance all procedures described by A. Mc Pherson, “Crystallization of Biological Macromolecules,” Cold Spring Harbor Laboratory Press, NY, 1999, the entire contents of which is incorporated herein by reference.

In a non-limiting manner, the following methods, particularly biochemical and/or physical methods, may be used or combined: salt fractionation, desalting, fractionation with organic solvents or with other precipitants, selection with heat/pH, centrifugation, chromatographic methods, e.g., ion exchange chromatography, molecular sieve chromatography, adsorption chromatography, affinity chromatography or HPLC, ultrafiltration, isoelectric focusing and/or electrophoresis by biochemical, particularly chromatographic, and/or physical methods.

Affinity chromatography is particularly preferred, whereby metals (e.g., Ni)and/or antibodies are typically bound to a resin as ligands. The affinity chromatography may typically be carried out in batch mode or by a column packed with an insoluble support matrix.

A further aspect of the present invention is a recombinant protein, particularly in isolated and/or purified form, obtainable from a method for producing of the recombinant protein of the invention as described herein.

A still further aspect of the present invention is a method for crystallizing a recombinant protein of the invention, wherein the method comprises (a) a purification step according to a method of the invention and (b) a crystallization step. Hereby, the purified recombinant protein obtained in step (a) is crystallized by any method known by the skilled person. The crystallizing step will be carried out under conditions suitable for crystal growth. The conditions may be optimized by varying certain parameters, such as stock solution, concentration of the recombinant protein, temperature, pH, ionic strength, precipitating agent (e.g., ammonium sulfate or PEG), addition of small amounts of organic solvents, etc. However, the conditions used for crystallization of component (1) alone are preferred, which means that the conditions suitable for a member of the myosin or kinesin superfamily or a fragment, analog or derivative thereof may also work to identify crystals of the recombinant protein of the invention.

In order to accelerate the crystallization process, it is particularly preferred to apply a recombinant protein of the invention containing as component (1) an amino acid sequence with a flexible region, particularly a flexible region at C-terminal end of (1). Thus, a high degree of flexibility of the components is achieved resulting in numerous conformations which can be occupied or sampled by the components in the course of the crystallization process.

It is preferred to employ vapor diffusion techniques either by the hanging or the sitting drop method to obtain crystals. Furthermore, crystallization may be achieved by induction of nucleation. Exemplary macro- or microseeding methods are described by A. Mc Pherson, “Crystallization of Biological Macromolecules,” Cold Spring Harbor Laboratory Press, NY, 1999, the contents of which is incorporated by reference.

Another aspect of the present invention is a protein crystal built by a network of recombinant proteins according to the invention. This network forms the crystal lattice. Within the scope of the present invention are crystals of any space group in which identical proteins can be arranged. A crystal of the invention may contain one, two, three or more recombinant proteins per asymmetric unit. At least one heavy atom may be located at a particular position or positions in the recombinant protein being arranged symmetrically in the crystal of the invention. Crystals may contain ligands non-covalently bound to the crystallized recombinant protein as well, e.g., ATP, inhibitors, alkali ions or physiological ligands, such as hormones, carbohydrates, protein fragments. etc.

Finally, an aspect of the present invention is a method for elucidating the atomic structure of a protein crystal of the invention, whereby, after a crystallization step (a) according to the invention, X-ray diffraction data are collected on a beamline or any kind of device suitable for measuring locations of X-ray reflections (diffractometer, (b)). In final step (c), the atomic structure or rather the electron density map (into which the polypeptide chain and, eventually, other ligands and water molecules are modeled) of a recombinant protein is calculated by Fourier transformation of the data set obtained in step (b) using phasing information obtained by anomalous scattering, the heavy atom method or molecular replacement techniques, as e.g., described by Stout & Jensen, X-ray Structure Determination, Wiley, NY, 1989, which is incorporated herein by reference.

For the present invention, molecular replacement methods are particularly useful. The phasing information may be obtained from component (1) as starting model, which is typically a structurally well determined polypeptide. Therefore, component (1) is a “helper” sequence providing the starting information to solve the structure of the recombinant protein or the structure of component (2), respectively, which is the target protein to be structurally analyzed. Further rounds of structure refinement by methods known by the skilled person or described by Stout & Jensen may serve to improve the structure model. Additionally, heavy atoms may be bound to known sites of component (1) of the recombinant protein of the invention. Thereby, additional phasing information may be obtained for structure elucidation of target component (2) (which is under analysis) of the recombinant protein of the invention.

The use of a recombinant protein of the invention for purification and crystallization purposes has unprecedented advantages over the methods known in the art. The recombinant protein via its component (1) binds to insoluble components of the cell, like the cytoskeleton, membrane components or the like. Following cell lysis, the recombinant fusion protein (or rather its component (2), which is the target protein desired to be purified, analyzed or subjected to X-ray analysis) can be enriched by ligand depletion and precipitation with the insoluble interaction partners of the cell. This allows for a purification step already carried out in the cell without any additional. Therefore, it is not the lysate as a whole which contains the overexpressed protein but the pre-purified precipitate itself. The specific solubilization of the fusion protein is achieved by addition of the ligand to the insoluble fraction.

For crystallization, the conditions (parameters) are preferably chosen such that they coincide with the conditions for structurally well characterized component (1). These conditions or subtle variations of these conditions are expected to work for the recombinant protein as well. Hence, the method of the present invention for crystallizing allows one to find crystallization conditions without extensive search for suitable parameters required by the art.

It is, however, within the scope of the present invention that a recombinant protein of the invention or any other recombinant protein which is purified according to a method of the present invention may be structurally analyzed by any other method known by the skilled person. Particularly, such recombinant proteins may be subjected to NMR analysis (two-dimensional or multidimensional) as described by Roberts, NMR of Macromolecules: A practical approach, Oxford-New York, 1993, which is incorporated herein by reference. Furthermore, the system of the present invention may be used for drug design (ligand to component (2) of the recombinant protein used) as described by Craik, NMR in Drug Design, CRC Press, Boca Raton, 1996, which is incorporated by reference. Other methods of structure eclucidation are, for instance, mass spectrotometry as described by Siuzdak, Mass Spectrometry for Biotechnology, Academic Press, San Diego, 1996, incorporated herein by reference.

Another aspect of the present invention is a method for isolating and identifying proteins that are capable of binding to the target protein sequence (component (2)) in the recombinant protein (particularly of the invention). Therefore, a yeast-two-hybrid system may be used, by which a sequence encoding the recombinant protein is carried by one hybrid vector and sequence carried by the second hybrid vector, the vectors being used to transform yeast host cells and the positive transformed cells being isolated, followed by extraction of the said second hybrid vector to obtain a sequence encoding a protein which binds to said recombinant protein directly or indirectly via other proteins.

Yet another aspect of the invention provides an approach/method suitable for the identification of binding partners to the recombinant protein of the invention. The method may comprise the following steps:

(a) a library of cDNA is typically fused to the C-terminus of (1), particularly of a myosin motor domain (MMD), (typically resulting in a recombinant protein of the invention), eventually via a linker sequence;

(b) the recombinant protein is expressed in Dictyostelium or other eukaryotic system;

(c) clonal transformants are probed with the bait-protein of choice fused to any marker protein, e.g., β-galactosidase; and

(d) after washing, identification and determination of interacting recombinant protein by measuring the activity of bait marker fusion protein, e.g., by addition of β-gal.

In a preferred embodiment, the myosin motor domain of step (a) may be His or epitope-tagged at the N-terminus. Typically all steps of this method of the invention are carried out in microtiter well plates.

Preferably, the recombinant protein shown to have bound to the bait-protein of choice may be purified by the methods of the invention and can then be subjected to further biochemical or structural characterization, e.g. crystallization as described above, with or without cleavage by a protease, if a recognition in a linker region has been provided, in order to release component (2), the target protein. This aspect of the invention is suitable for the identification of unknown binding partners and may also be used to demonstrate the interaction between two known polypeptides.

The disclosed method of isolating yet unknown binding partners of the invention has numerous advantages over methods known in the art. MMD-fusion proteins, for example, may be easily purified from Dictyostelium and the MMD fusion system may be transferred to a wide range of high eukaryotic cells. Further advantages include: (i) the MMD-cDNA constructs may be directly used for expression in Dictyostelium and other eukaryotic cells; (ii) decreased background (since the system works with purified proteins and not with proteins within a cellular environment that, as in the case of the yeast 2-hybrid-system, leads to a high background of false positive clones); (iii) easy identification and isolation of the positive construct from mother plates; and (iv) the procedure may be highly automated, since all steps in the interaction screening may be performed in microtiter well plates.

### BRIEF DESCRIPTION OF THE DRAWINGS

FIGS. 1A and 1B show the structure of M761-2R-R238E, an example for a recombinant protein of the invention. Although two molecules are present in the crystallographic asymmetric unit, only one is shown here. The two molecules are essentially identical throughout the myosin motor domain (residues 2-761) exemplifying component (1) of the recombinant protein of the invention. However, upon leaving the converter domain, the lever arms assume slightly different orientations and deviate at the ends by 19.4 Å.

FIG. 1A shows a complete molecule (recombinant protein of the invention) spanning amino acids 2-1010. No electron density was observed for five residues at the N-terminus, the loop region 205-208, and one residue at the C-terminus. The molecule comprises the N-terminal domain (2-200), 50 kDa domain (201-613), C-terminal and converter domain (614-761), linker region (762-764) (component (3)) of the recombinant protein of the invention), α-actinin lever arm (765-1003) (component (2)) of the recombinant protein of the invention) and seven histidines from the His purification tag (1004-1010), which are linked as specified for an preferred embodiment of the present invention. The linker region (3) is composed of three residues (Leu-Gly-Arg) introduced during cloning. The observed lever arm is ˜140 Å long (measured from Cα of 761 to Cα of 1010). Each α-actinin repeat contributes ˜65 Å, and the histidine purification tag another 10 Å. Helices 1-3 make up the first α-actinin repeat, and 4-6 the second. The arrowhead indicates the α-helical region linking the two repeats. The disruptive kink in helix 2 is caused by the presence of two adjacent proline residues (FIG. 5A).

FIG. 1B is a detailed view of the linker region joining the myosin converter domain to helix 1 of α-actinin. The view is rotated 180° around a vertical axis from FIG. 1A.

FIGS. 2A, 2B and 2C provide a detailed view of the conserved salt bridge linking switch I and switch II as a result of purifying, crystallizing a recombinant protein of the invention and finally solving the structure of that protein according to methods of the invention. The conserved nucleotide binding/sensing elements found in all myosins, kinesins, and G-proteins include the P-loop, switch I, and switch II.

FIG. 2A shows the structure of Dictyostelium myosin II motor complexed with Mg-ADP-BeF₃. As in Mg-ADP-VO₄ (Smith and Rayment, 1996a) and Mg-ADP-BeF₃ (Dominguez et al., 1998) structures, switch I and switch II are closed. The conserved salt bridge between residues R238 and E459 is shown as a ball-and-stick model surrounded by 2.6 Å experimental 2fₒ-f_{c} electron density (wireframe), contoured at 1σ. As expected for a salt bridge, the electron density is continuous between the residues, which point toward each other.

FIG. 2B is the same region as observed in the crystal structure of M761-2R-R238E. The electron density was calculated from a model with alanins at positions 238 and 459 in order to eliminate model bias. Electron density for two glutamic acid residues is clearly visible, but the side chain of E238 now points away from E459 and the switch II loop has moved away from switch I.

FIG. 2C again illustrates the same region showing a superposition of the M761-2R-R238E structure with a structure of Dictyostelium, myosin II motor complexed with Mg-ADP-VO₄ (PDB code IVOM) (Smith and Rayment, 1996a). The nucleotide and R238-E459 salt bridge are shown as ball-and-stick models. Both the P-loop and switch I regions are in essentially identical conformations in both structures. However, the switch II region shifts to the right, toward the nucleotide, by ˜Å in the Mg-ADP-VO₄ structure, allowing the formation of the R238-E459 salt bridge.

FIG. 3 shows the orientation of the myosin lever arm, a segment of component (1) of an example for an recombinant protein of the invention. Shown are five molecules of actin making up part of a helical actin filament. Modeled onto this structure are myosin in the “pre-power stroke” up/closed orientation, the “post-power stroke” down/open orientation, and the M761-2R-R238E structure. First, the up, down, and actomyosin complex structures were modeled, and the M761-2R-R238E structure was then aligned to the core domain of the down/open structure via residues 160-200, which includes the highly conserved P-loop region. It is noted that in the M761-2R-R238E structure, the helix leaving the converter domain initially superposes with the down/open structure, but then deviates due to the different helical bend of the α-actinin.

FIGS. 4A and 4B depict the structure of α-actinin repeats 1 and 2. α-Actinin is an example for component (2) of the recombinant protein of the present invention, which means α-actinin is the target protein in this example. Its structure was solved using purification and crystallization methods of the present invention.

FIG. 4A shows an α-carbon chain trace of the 6 helices making up repeats 1 (helices labeled 1-3) and 2 (helices labeled 4-6). The 17 hydrophobic aromatic amino acid residues stabilizing the triple-helical packing include 7 tyrosines, 6 phenylalanines and 4 tryptophans. Shown also are two adjacent proline residues, which cause a kink, but not a break in α-helix 2 of repeat 1. The uninterrupted α-helix linking repeats 1 and 2 is shown.

FIG. 4B is a detailed view of the linker region, highlighting the stabilizing hydrophobic and hydrogen bonding interactions. Orientation is identical to that in FIG. 4A. Side chains are shown as ball-and-stick models, with the exception of Asp796 and Ser797, in which only the α-carbon atoms involved in hydrophobic contacts are shown for clarity. The salt bridge between Arg880 and Glu877, and the hydrogen bond between Arg880 and the carbonyl oxygen of Leu956 (also shown as a ball-and-stick model), are shown as dashed lines.

FIGS. 5A and 5B provide a comparison of Dictyostelium α-actinin with human α-actinin and human α-spectrin.

FIG. 5A shows the overlapping repeat 2 region of Dictyostelium and human α-actinin as ribbon diagrams. Helices are numbered as described above for Dictyostelium α-actinin and, in parentheses, as described previously for human α-actinin (Djinovic-Carugo et al., 1999). The largest differences occur in the loop region connecting helices 4 and 5, indicated by an arrow, where human α-actinin would seriously overlap with Dictyostelium helix 6.

FIG. 5B shows the alignment of Dictyostelium repeat 2 with repeat 16 human α-spectrin as ribbon diagrams. Helices are numbered as described above for the Dictyostelium protein and, in parentheses, as described previously for the human protein (Gram et al., 1999). Dictyostelium helix 4 and α-spectrin helix A are in the background. In general, the two structures align more closely than the human/Dictyostelium alignment described in FIG. 5A. The largest difference occurs in the loop region connecting helices 5 and 6, indicated by an arrow, where the human α-spectrin structure is moved in respect to the Dictyostelium α-actinin structure as a result of a proline-induced kink in helix B.

FIG. 6 provides the amino acid sequence (one-letter-code) for component (1) of recombinant protein M761-2R-R238E, exemplifying a recombinant protein of the invention. This sequence is further illustrated as attached SEQ ID NO. 1.

FIG. 7 provides the whole sequence of recombinant protein M761-2R-R238E comprising as component (1) the amino acid sequence of the myosin II motor domain of Dictyostelium, a three amino acid linker region (LGS) as component (3) and the α-actinin amino acid sequence being the target sequence (component (2)) in this example (one-letter-code). This sequence is further illustrated as attached SEQ ID NO. 2.

FIG. 8 is the DNA sequence coding for recombinant protein M761-2R-R238E such that the sequence of FIG. 8 corresponds to the sequence of FIG. 7 on the genetic level. This sequence is further illustrated as attached SEQ ID NO. 3.

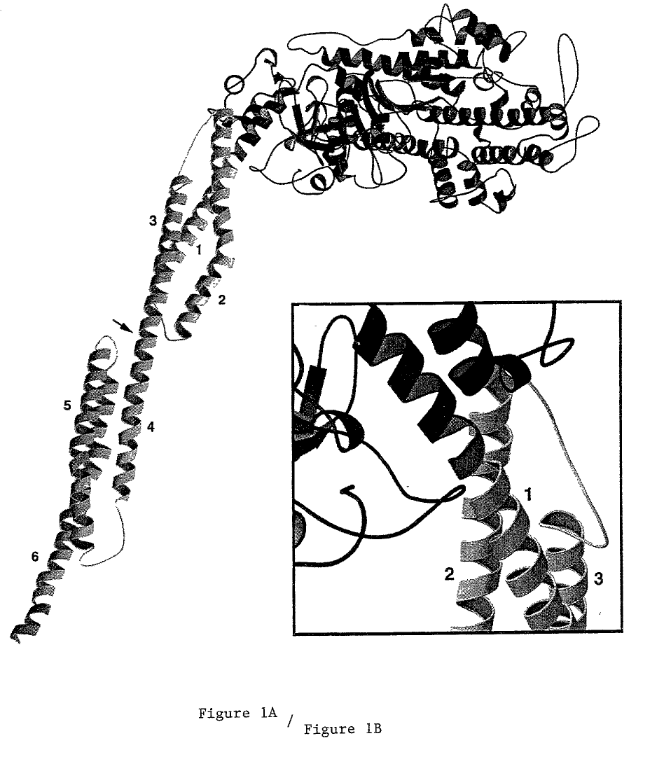

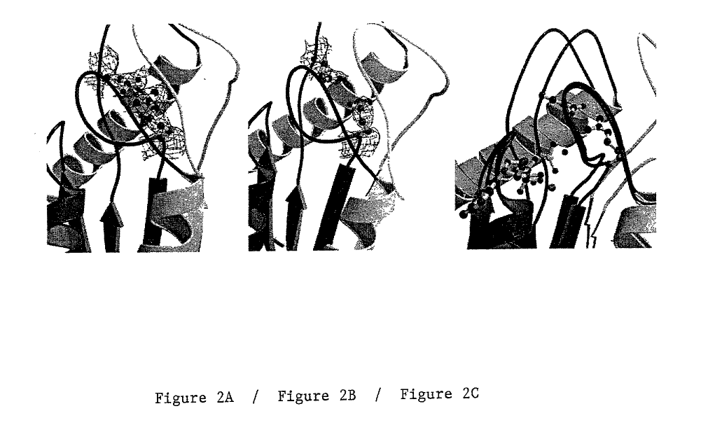

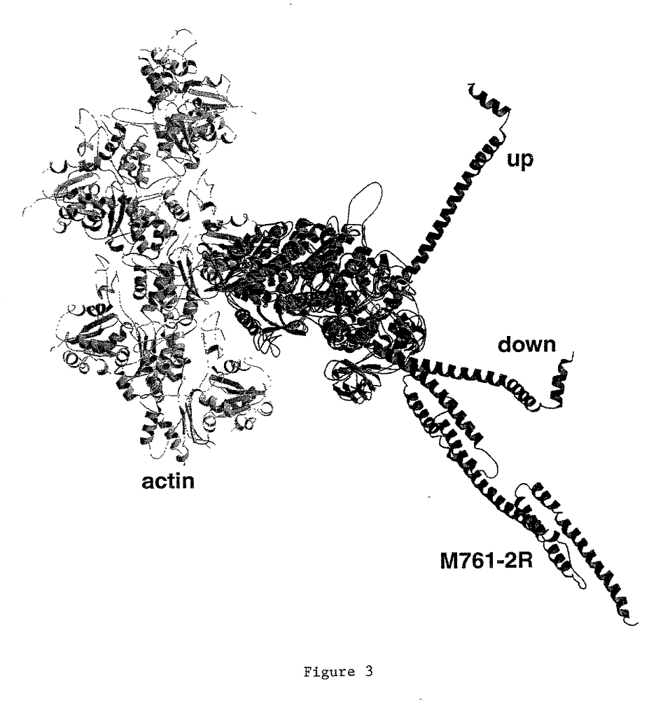

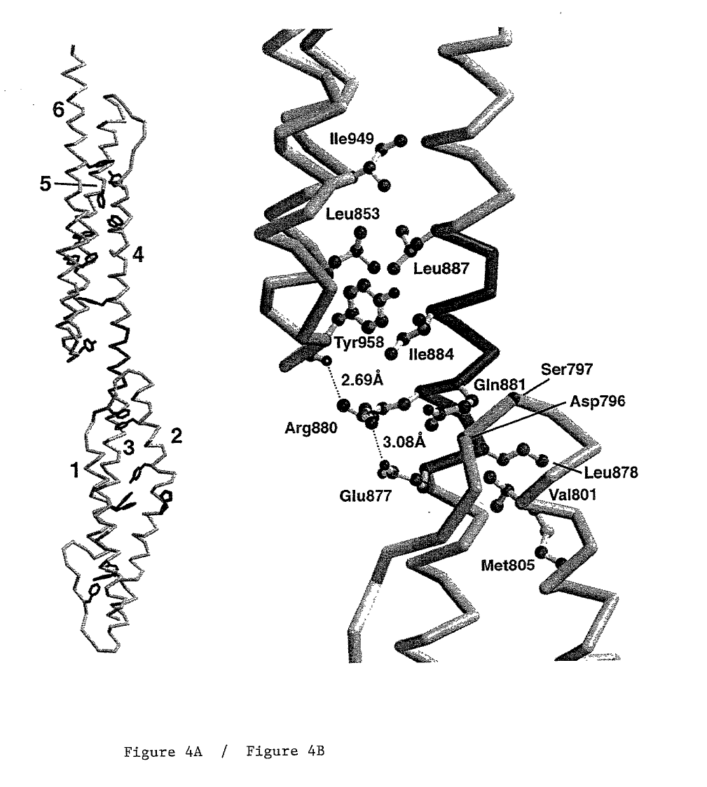

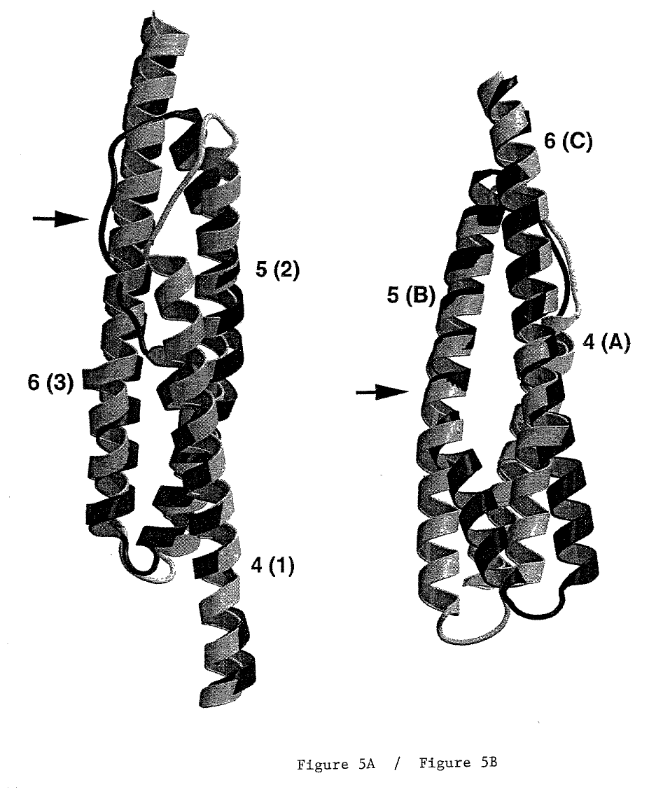

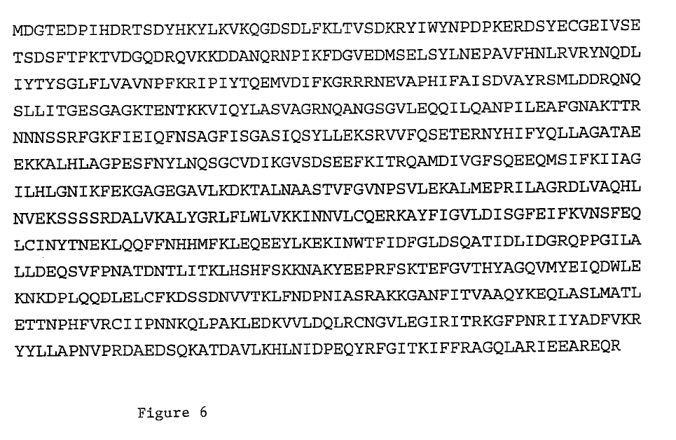

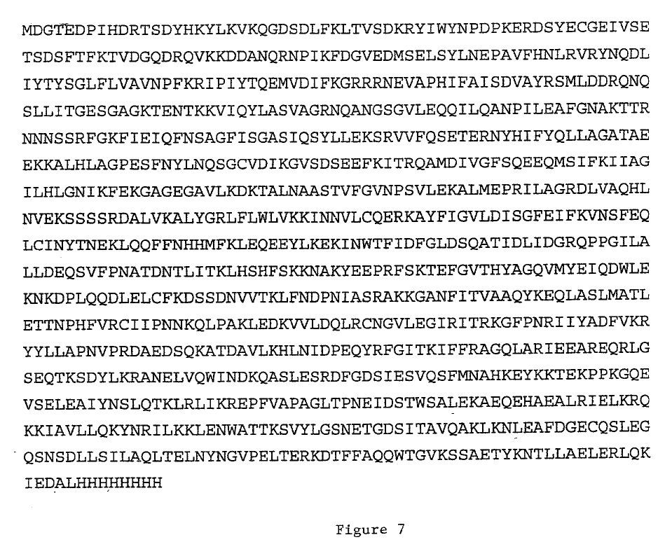

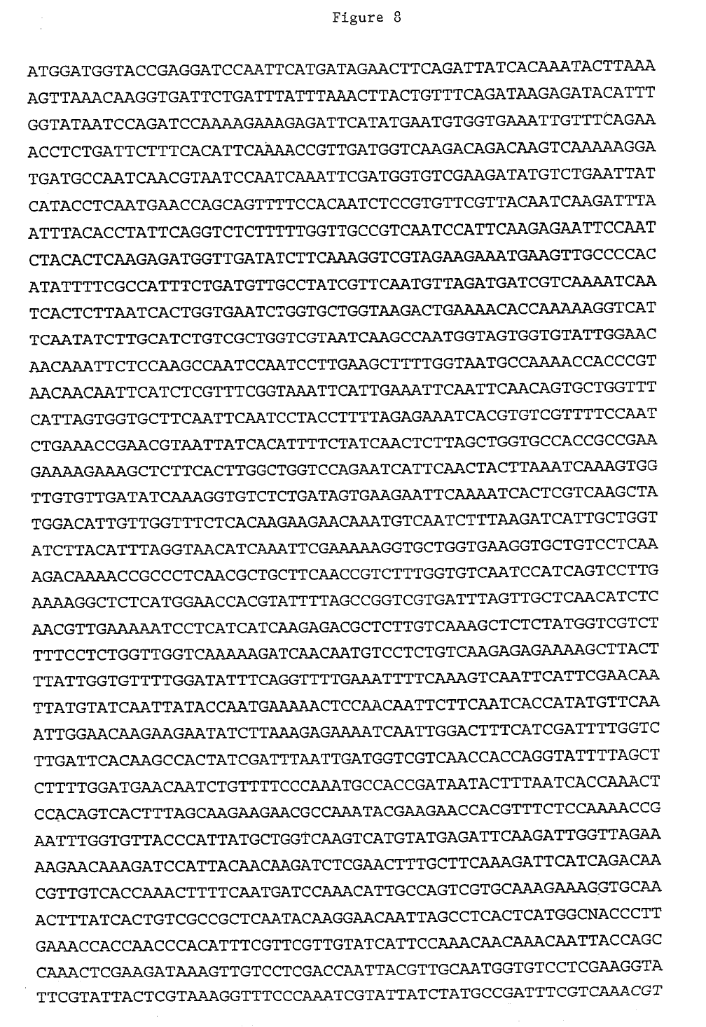

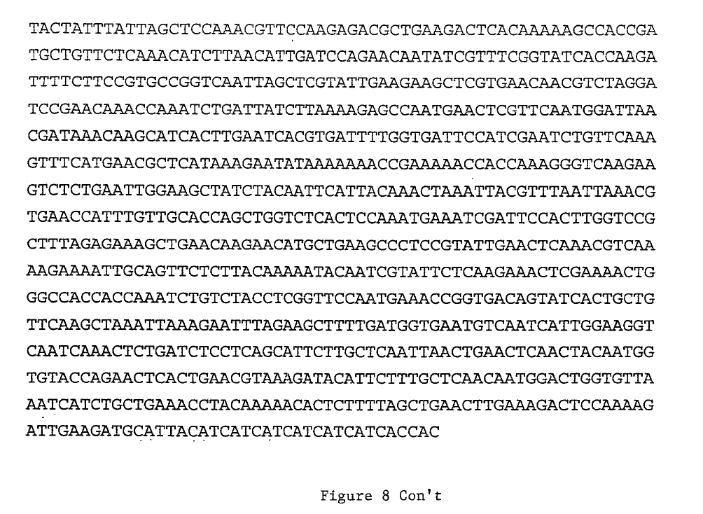

### DETAILED DESCRIPTION OF THE INVENTION

### Example 1

(a) Expression

The expression-vector pDXA-3H, that was used for the production of M761-2R-R238E, carries the origin of replication of the Dictyostelium high copy number plasmid Ddp2 (Leiting et al., Molecular And Cellular Biology, 10:3727-3736, 1990; Chang et al., Nucleic Acids Research, 17:3655-3661, 1990), an expression cassette consisting of the strong, constitutive actin15 promoter, a translational start codon upstream from a multiple cloning site (MCS), and sequences for the addition of a histidine octamer at the carboxy terminus of any protein. Plasmids derived from pDXA-3H were transformed into orf⁺-cells. These cells carry several integrated copies of the rep gene which is essential in trans for the replication of plasmids that carry the Ddp2 origin (Leiting et al., Molecular And Cellular Biology, 10:3727-3736, 1990; Slade et al., Plasmid, 24:195-207, 1990). The myosin-α-actinin fusion was created by linking codon 761 of the Dictyostelium mhcA gene to codon 264 of the Dictyostelium α-actinin gene.

The resulting construct pDH12-2R extended to codon 505 of the α-actinin gene. Plasmid pDH20 was generated by insertion of the first 765 codons of Dictyostelium myosin II into the MCS of pDXA-3H (Furch et al., Biochemistry, 37:6317-6326, 1988). Site directed mutagenesis was used to generate plasmid pDH20(R238E) encoding a motor domain fragment with the single point mutation R238E. Replacement of the 2 kb SafI-BstXI fragment of pDH12-2R with the corresponding fragment from pDH20(R23BE) was used to generate the expression vector for the production of M761-2R-R238E, the fusion protein and an example for a recombinant protein of the invention, thus containing both a point mutation in the active site and a C-terminal extension consisting of two α-actinin repeats.

(b) Purification

The overexpressed protein was purified by Ni²⁺-chelate affinity chromatography as described by Manstein and Hunt, J. Muscle R. Cell Motil., 6:325 1995 and Manstein et al., Gene, 162:129, 1995. The entire contents of each of which is incorporated herein by reference.

Cells expressing the histidine octamer tagged fusion protein were grown in 5 L flasks containing 2.5 L DD-Broth 20. DD-Broth 20 contains (per liter): 20 g protease peptone (Oxoid), 7 g yeast extract (Oxoid), 8 g glucose, 0.33 g Na₂HPO₄.7H₂O, and 0.35 g KH₂PO₄. The flasks were incubated on a gyratory shaker at 200 rpm and 21° C. Cells were harvested at a density of 6×10⁶ ml⁻¹ by centrifugation for 7 min at 2,700 rpm in a Beckman J-6 centrifuge and washed once in PBS. The wet weight of the resulting cell pellet was determined. Typically, 35 g were obtained from a 15 L shaking culture. The cells were resuspended in 140 ml of Lysis Buffer (50 mM Tris-HCl, pH 8.0, 2 mM EDTA, 0.2 mM EGTA, 1 mM dithiothreitol (DTT), 5 mM benzamidine, 40 mg/ml TLCK, 20 mg/ml N-tosyl-L-phenylalanine chloromethyl ketone (TPCK), 200 mM phenylmethylsulfonyl fluoride (PMSF) and 0.04% NaN₃).

Cell lysis was induced by the addition of 70 ml of Lysis Buffer containing 1% Triton-X®100, 15 mg/ml RNaseA (Sigma) and 100 units of alkaline phosphatase. The lysate was incubated on ice for one hour. Upon centrifugation (230,000 g, 1 hour), the recombinant protein remained in the pellet. The pellet was washed in 100 ml of HKM buffer (50 mM HEPES, pH 7.3, 30 mM KAc, 10 mM MgSO₄, 7 mM b-mercaptoethenol, 5 mM benzamidine, 40 mg/ml PMSF) and centrifuged for 45 min at 230,000 g. The recombinant protein was released into the supernatant by extraction of the resulting pellet with 60 ml HKM buffer containing 10 mM ATP. After centrifugation (500,000 g, 45 min.), the supernatant was loaded using a peristaltic pump onto a Ni²⁺-nitrilotriacetic acid (Ni²⁺-NTA) affinity column (1.5×10 cm) (Qiagen). The flow-rate was adjusted to approximately 3 ml min⁻¹. After loading was completed the column was connected to a Waters 650M chromatography system. The column was washed briefly in Low Salt buffer (50 mM HEPES, pH 7.3, 30 mM KAc, 3 mM benzamidine), High Salt buffer (as Low Salt Buffer, but with 300 mM KAc), and Low Salt Buffer containing 50 mM imidazole. The recombinant myosin was eluted using a linear gradient of Low Salt Buffer and Imidazole Buffer (0.5 M imidazole, pH 7.3, 3 mM benzamidine), starting with 10% Imidazole Buffer and reaching 100% after 15 minutes. The flow rate was 3 ml min⁻¹ and 3 ml fractions were collected. Absorbance at 280 nm was monitored. SDS gels were run to check the purity of the eluted protein.

The pooled fractions were dialyzed immediately against storage buffer (20 mM HEPES, 0.5 mM EDTA, 1 mM DTT, pH 7.0) containing 3% sucrose and the purified protein could be stored at −80° C. for several months without apparent loss of enzymatic activity. Actin-activated ATPase activity was measured by the release of inorganic phosphate.

(c) Crystallization

Crystals of the overexpressed and purified recombinant protein M761-2R-R238E were grown by the hanging drop method at 7° C. The drops contained equal volumes (2.2 μl) of the protein solution and the mother liquor. The mother liquor contained 12% PEGM 5K, 170 mM NaCl, 50 mM HEPES-NaOH pH 7.2, 5 mM MgCl₂, 5 mM DTT, 0.5 mM EGTA and 2% 2-methyl-1,3-propanediol. The protein solution (5 mg/ml) contained additionally 200 μM ADP and 200 μM vanadate, and was incubated on ice for 1 h before setting up the drops. Crystals normally appeared after 7-8 days and reached maximum dimensions of 0.1×0.3×0.9 mm. Crystals were transferred to a solution of mother liquor plus 30% glycerol and frozen in liquid nitrogen for storage and data collection.

(d) Crystallography and structure refinement

Diffraction data for the crystals of the recombinant protein M761-2R-R238E were collected at ESRF beamline ID-13 on a MarCCD detector and integrated and scaled using the program XDS (Kabsch, J. Appl. Cryst., 26:795, 1993), producing a data set 97.7% complete to 2.8 Å with 4-fold redundancy and an R_{sym} of 11.0%. The M761-2R-R238E crystals belonged to space group P2,2,2 with two molecules in the asymmetric unit. Molecular replacement was performed with the program AMoRe (Navaza, Acta Cryst. A, 50:57, 1994) using the crystal structure of Dictyostelium myosin resides 2-759 complexed with Mg-ADP-BeFₓ (PDB code lmmd) (Fisher et al., Biochemistry, 34:8960, 1995) as a starting model (the nucleotide and the side chains beyond Cβ of residues 238 and 459 were excluded).

Initial maps showed clear helical density for the first repeat of the α-actinin lever arm, which was built as a poly-alanine model using the program O (7.0 for WindowsNT), Jones et al., “Improved methods for building protein models in electron density maps and the location of errors in these models,” Acta Crystallogr. A, 47L110-119, 1991. Following several rounds of simulated annealing refinement using torsional dynamics and a maximum likelihood target with the program CNS v0.9a (Brünger et al., 1998, Acta Cryst. D, 54:905), the second α-actinin repeat was visible and built. Subsequent rounds of model building and refinement (including bulk solvent correction) produced the final structure of two M761-2R-R238E molecules containing 1005 residues each, two molecules of Mg-ADP and 14 water molecules (R-factor, 24.1%; R_{free}, 29.9%). Ramachandran analysis shows all nonglycine residues to be in allowed regions. Figures were made using the programs Bobscript (Esnouf, J. Mol. Graph. Model., 15:132, 1997) and Raster3D (Merritt and Bacon, Methods Enzymol., 277:505, 1997).

In contemplation of the principles of the present invention, reference is made to Niemann et al., “Crystal structure of a dynamin GTPase domain in both nucleotide-free and GDP-bound forms,” EMBO Journal, 20:5813-5821, 2001 and Kliche et al., “Structure of a genetically engineered molecular motor.” EMBO Journal, 20:40-46, 2001.

TABLE 1

| | | | | | | Structure | Thrombin |
|---|---|---|---|---|---|---|---|
| Protein | Fusion Partner | Transformed | Expressed | Purified | Crystallized | Solved | Cleavage |
| M761-2R | Repeats 1 and 2 of D.discoideum α-actinin | Y | Y | Y | Y | Y | X |
| M765-DymA2-316 | D.discoideum Dynamin A residues 2-316 | Y | Y | Y | Y | Y | N |
| M765-DymA2-490 | D.discoideum Dynamin A residues 2-490 | Y | Y | N | X | X | X |
| M765-SSF | D.discoideum SSF protein - unknown function | Y | Y | Y | N | X | Y |
| M765-Mark1-A2 | Mammalian mutant Mark1 kinase domain | Y | Y | Y | N | X | Y |
| M765-Mark2 | Mammalian Mark1 kinase domain | Y | Y | X | X | X | X |
| M765-Kif2c632 | Mammalian kinesin related protein domain | Y | Y | X | X | X | X |
| M765-Kif2-md | Mammalian kinesin related protein domain | Y | Y | X | X | X | X |
| M765-kiffuII | Mammalian kinesin related complete protein | Y | X | X | X | X | X |
| M765-jmjmC | D.discoideum universal transcriptional regulator | Y | X | X | X | X | X |
| M765-p27 | P27 protein | Y | N | X | X | X | X |
| M765-HsCor1A | Human coronin protein | Y | N | X | X | X | X |
| M765-DdCor | D.discoideum coronin protein | Y | Y | X | X | X | X |
| M765-IRT1 | Arabadopsis metal transport protein | Y | N | X | X | X | X |
| M765-DdNck2 | D.discoideum Nck protein | Y | N | X | X | X | X |

Y - Tested and worked
N - Tested and did not work
X - Not tested

### Example 2

Myosin-Fusion-System for isolating interacting proteins/protein binding partners

(a) Preparation

In order to demonstrate the function of the myosin-fusion-system a library of cDNA was fused to the C-terminus of an MMD and expressed in Dictyostelium or another eukaryotic system. Clonal transformants were probed with the bait-protein of choice fused to β-galactosidase. The MMD was His- or epitope-tagged at the N-terminus.

Experimentally, cells were transformed with the MMD-cDNA library and clones were grown and kept in 96 well plates. The bait-β-gal fusion protein was transformed in Dictyostelium orf⁺ cells and grown in an appropriate quantity (1 clonal cell line). Upon reaching confluence, the MMD-cDNA clones in the 96 well plates were washed once in the plates with PBS and then lysed by adding lysis buffer containing Triton X®-100 (or, alternatively, NP-40), at the same time the ATP pool was depleted by the addition of alkaline phosphatase. The actin-based cytoskeleton with all myosin and also the M765-fusion-proteins were pelleted by centrifugation and washed with lysis buffer. The myosin was released from the pellets by the addition of Mg²⁺-ATP. The ATP-unsoluble fraction was pelleted and the supernatant transferred to 96 well plates coated with Ni-NTA. The His-tagged products of the MMD-cDNA were shown to bind to these plates. After extensive washing, the coated plates were incubated with the bait-β-gal construct. Again, after extensive washing, the plates were incubated with a substrate for β-gal, in this case CPRG (red color OD₅₇₄) or ONPG (yellow OD₄₁₅), and the β-gal activity was determined with a microtiter plate reader. High β-gal activity indicated a strong interaction between the bait and the product of the target cDNA.

The selected clones were then recovered from the original 96 well plates. The MMD-cDNA-clone was expressed in and purified from Dictyostelium by standard MMD purification. For further biochemical and structural characterization, the isolated gene product was either cleaved with an appropriate protease to release it from the MMD or was used directly in the fusion form for kinetics or crystallization experiments.

(b) Interaction Test

The method of the invention was tested by expressing MMD-RaclA and DRG-2D-β-gal (the DRG-2D construct acts as an exchange factor for the small G-protein RaclA).

The MMD-RaclA cells were cloned, grown in 96 well plates, washed, lysed and ATP extracted as described above. The Ni-NTA coated plates were then incubated with the ATP-released protein fraction. The cells expressing the DRG-2D-β-gal were grown in shaking suspension and washed and lysed under the same conditions. The DRG-2D-β-gal supernatant was incubated at different dilutions. As control wells were incubated without bait (DRG-2D-β-gal) or without MMD-RaclA or with MMD alone, all controls were negative after staining for β-gal, whereas the incubations with immobilized MMD-RaclA and the bait gave a signal, which was dependent on the concentration of added bait.

In conclusion, the interaction between DRG-2D and RaclA was shown by the method of the invention, whereas it could not be shown when using the yeast-two-hybrid system. Therefore, the method of the invention has definite advantages over the yeast-two-hybrid system or other known techniques developed to identify protein-protein interactions.

This invention has been described in terms of specific embodiments, set forth in detail. It should be understood, however, that these embodiments are presented by way of illustration only, and that the invention is not necessarily limited thereto.

We claim:

1. A recombinant protein comprising:
(a) a first protein, or an analog, fragment or derivative thereof; and
(b) a target protein of interest.

2. The recombinant protein of claim 1, further comprising:
(c) a linker between (a) and (b).

3. The recombinant protein of claim 2, wherein (c) comprises at least 2 amino acids.

4. The recombinant protein of claim 1, wherein (b) is any amino acid sequence of at least 20 amino acids.

5. The recombinant protein of claim 4, wherein (a) comprises an amino acid sequence of a member of the myosin or kinesin protein superfamilies, or an analog, fragment or derivative thereof.

6. The recombinant protein of claim 5, wherein (a) is chosen from the group consisting of amino acid sequences of a member of the myosin I, II, III, IV, V, VI, VIII, X or XI families, or the kinesin I or II families, or an analog, fragment derivative thereof.

7. The recombinant protein of claim 5, wherein (a) is an amino acid sequence for the motor domain of a member of the myosin or kinesin protein superfamilies, or an analog, fragment or derivative thereof.

8. The recombinant protein of claim 3, wherein (c) comprises a sequence of 3 amino acids, wherein Gly is in the second position.

9. The recombinant protein of claim 4, wherein (b) comprises the amino acid sequence of an esterase, hydrolase, phosphatase, kinase, protease, channel, structural protein, receptor, transcription factor, DNA/RNA-binding protein, lipoprotein or glycoprotein, or an analog, derivative or fragment thereof.

10. The recombinant protein of claim 9, wherein (b) is selected from the group consisting of the structural proteins coronin or spectrin, and a neuronal or immunologically relevant receptor.

11. The recombinant protein of claim 1, wherein (a) comprises an amino acid sequence of SEQ ID NO. 1.

12. The recombinant protein of claim 11, wherein (c) comprises the amino acid sequence Leu-Gly-Ser.

13. The recombinant protein of claim 1, further comprising a tag sequence at the N- or C-terminus of the protein.

14. A DNA sequence comprising an amino acid sequence that codes for the recombinant protein of claim 1.

15. An expression vector comprising the DNA sequence of claim 14.

16. An expression vector of claim 15, capable of expression in an eukaryotic host cell.

17. The expression vector of claim 16, capable of expression in cells of Dictyostelium.

18. A transformed eukaryotic host cell comprising a vector of claim 16.

19. A transformed eukaryotic host cell comprising a vector of claim 17.

20. A method for producing a recombinant protein according to claim 1, the method comprising the steps of:
(a) preparing an expression vector comprising a DNA sequence that codes for the recombinant protein of claim 1;
(b) transforming eukaryotic host cells with a vector obtainable from step (a); and
(c) growing transformed host cells obtainable from step (b) under conditions suitable for the expression of said recombinant protein.

21. A method for purifying a recombinant protein according to claim 1, the method comprising the steps of:
(a) preparing an expression vector comprising a DNA sequence that codes for the recombinant protein of claim 1;
(b) transforming eukaryotic host cells with a vector obtainable from step (a);
(c) growing transformed host cells obtainable from step (b) under conditions suitable for the overexpression of said recombinant protein;
(d) purifying overexpressed recombinant protein by binding to endogenous actin or microtubules of the eukaryotic host cell; and
(e) specifically releasing bound recombinant protein from the actin or microtubules.

22. The method of claim 21, wherein (e) comprises releasing the recombinant protein by adding a natural substrate of component (a) of claim 1.

23. The method according to claim 22, wherein the natural substrate is ATP.

24. The method of claim 21, further comprising at least one additional purifying step, chosen from biochemical, chromatographic and physical methods, or combinations thereof.

25. The method of claim 21, wherein the additional purification step comprises affinity chromatography.

26. The method of claim 25, wherein the affinity chromatography utilizes metals or antibodies as ligands.

27. A method for crystallizing a recombinant protein, the method comprising the steps of:
(a) purifying the recombinant protein according to the method of claim 21; and
(b) crystallizing the purified recombinant protein obtained in step (a).

28. A protein crystal having a crystal lattice formed by a network of recombinant proteins of claim 1.

29. A method for elucidating the atomic structure of a protein crystal, the method comprising the steps of:
(a) crystallizing a recombinant protein according to the method of claim 27;
(b) collecting X-ray diffraction data for the protein crystal obtained in step (a); and
(c) calculating the atomic structure of the recombinant protein by transformation of the data obtained in step (b).


SEQUENCE LISTING (3 sequences)

SEQ ID NO: 1 - protein, 765 aa, Artificial Sequence
  Description of Artificial Sequence Partial myosin sequence of Dictyostelium; Component (1) of the recombinant protein M761-2R  R238E
MDGTEDPIHDRTSDYHKYLKVKQGDSDLFKLTVSDKRYIWYNPDPKERDSYECGEIVSETSDSFTFKTVDGQDRQVKKDDANQRNPIKFDGVEDMSELSYLNEPAVFHNLRVRYNQDLIYTYSGLFLVAVNPFKRIPIYTQEMVDIFKGRRRNEVAPHIFAISDVAYRSMLDDRQNQSLLITGESGAGKTENTKKVIQYLASVAGRNQANGSGVLEQQILQANPILEAFGNAKTTRNNNSSRFGKFIEIQFNSAGFISGASIQSYLLEKSRVVFQSETERNYHIFYQLLAGATAEEKKALHLAGPESFNYLNQSGCVDIKGVSDSEEFKITRQAMDIVGFSQEEQMSIFKIIAGILHLGNIKFEKGAGEGAVLKDKTALNAASTVFGVNPSVLEKALMEPRILAGRDLVAQHLNVEKSSSSRDALVKALYGRLFLWLVKKINNVLCQERKAYFIGVLDISGFEIFKVNSFEQLCINYTNEKLQQFFNHHMFKLEQEEYLKEKINWTFIDFGLDSQATIDLIDGRQPPGILALLDEQSVFPNATDNTLITKLHSHFSKKNAKYEEPRFSKTEFGVTHYAGQVMYEIQDWLEKNKDPLQQDLELCFKDSSDNVVTKLFNDPNIASRAKKGANFITVAAQYKEQLASLMATLETTNPHFVRCIIPNNKQLPAKLEDKVVLDQLRCNGVLEGIRITRKGFPNRIIYADFVKRYYLLAPNVPRDAEDSQKATDAVLKHLNIDPEQYRFGITKIFFRAGQLARIEEAREQR

SEQ ID NO: 2 - protein, 1016 aa, Artificial Sequence
  Description of Artificial Sequence Whole sequence of recombinant protein M761-2R R238 E
MDGTEDPIHDRTSDYHKYLKVKQGDSDLFKLTVSDKRYIWYNPDPKERDSYECGEIVSETSDSFTFKTVDGQDRQVKKDDANQRNPIKFDGVEDMSELSYLNEPAVFHNLRVRYNQDLIYTYSGLFLVAVNPFKRIPIYTQEMVDIFKGRRRNEVAPHIFAISDVAYRSMLDDRQNQSLLITGESGAGKTENTKKVIQYLASVAGRNQANGSGVLEQQILQANPILEAFGNAKTTRNNNSSRFGKFIEIQFNSAGFISGASIQSYLLEKSRVVFQSETERNYHIFYQLLAGATAEEKKALHLAGPESFNYLNQSGCVDIKGVSDSEEFKITRQAMDIVGFSQEEQMSIFKIIAGILHLGNIKFEKGAGEGAVLKDKTALNAASTVFGVNPSVLEKALMEPRILAGRDLVAQHLNVEKSSSSRDALVKALYGRLFLWLVKKINNVLCQERKAYFIGVLDISGFEIFKVNSFEQLCINYTNEKLQQFFNHHMFKLEQEEYLKEKINWTFIDFGLDSQATIDLIDGRQPPGILALLDEQSVFPNATDNTLITKLHSHFSKKNAKYEEPRFSKTEFGVTHYAGQVMYEIQDWLEKNKDPLQQDLELCFKDSSDNVVTKLFNDPNIASRAKKGANFITVAAQYKEQLASLMATLETTNPHFVRCIIPNNKQLPAKLEDKVVLDQLRCNGVLEGIRITRKGFPNRIIYADFVKRYYLLAPNVPRDAEDSQKATDAVLKHLNIDPEQYRFGITKIFFRAGQLARIEEAREQRLGSEQTKSDYLKRANELVQWINDKQASLESRDFGDSIESVQSFMNAHKEYKKTEKPPKGQEVSELEAIYNSLQTKLRLIKREPFVAPAGLTPNEIDSTWSALEKAEQEHAEALRIELKRQKKIAVLLQKYNRILKKLENWATTKSVYLGSNETGDSITAVQAKLKNLEAFDGECQSLEGQSNSDLLSILAQLTELNYNGVPELTERKDTFFAQQWTGVKSSAETYKNTLLAELERLQKIEDALHHHHHHHH

SEQ ID NO: 3 - DNA, 3048 bp, Artificial Sequence
  Description of Artificial Sequence DNA sequence coding for recombinant protein M761-2R R238E
atggatggtaccgaggatccaattcatgatagaacttcagattatcacaaatacttaaaagttaaacaaggtgattctgatttatttaaacttactgtttcagataagagatacatttggtataatccagatccaaaagaaagagattcatatgaatgtggtgaaattgtttcagaaacctctgattctttcacattcaaaaccgttgatggtcaagacagacaagtcaaaaaggatgatgccaatcaacgtaatccaatcaaattcgatggtgtcgaagatatgtctgaattatcatacctcaatgaaccagcagttttccacaatctccgtgttcgttacaatcaagatttaatttacacctattcaggtctctttttggttgccgtcaatccattcaagagaattccaatctacactcaagagatggttgatatcttcaaaggtcgtagaagaaatgaagttgccccacatattttcgccatttctgatgttgcctatcgttcaatgttagatgatcgtcaaaatcaatcactcttaatcactggtgaatctggtgctggtaagactgaaaacaccaaaaaggtcattcaatatcttgcatctgtcgctggtcgtaatcaagccaatggtagtggtgtattggaacaacaaattctccaagccaatccaatccttgaagcttttggtaatgccaaaaccacccgtaacaacaattcatctcgtttcggtaaattcattgaaattcaattcaacagtgctggtttcattagtggtgcttcaattcaatcctaccttttagagaaatcacgtgtcgttttccaatctgaaaccgaacgtaattatcacattttctatcaactcttagctggtgccaccgccgaagaaaagaaagctcttcacttggctggtccagaatcattcaactacttaaatcaaagtggttgtgttgatatcaaaggtgtctctgatagtgaagaattcaaaatcactcgtcaagctatggacattgttggtttctcacaagaagaacaaatgtcaatctttaagatcattgctggtatcttacatttaggtaacatcaaattcgaaaaaggtgctggtgaaggtgctgtcctcaaagacaaaaccgccctcaacgctgcttcaaccgtctttggtgtcaatccatcagtccttgaaaaggctctcatggaaccacgtattttagccggtcgtgatttagttgctcaacatctcaacgttgaaaaatcctcatcatcaagagacgctcttgtcaaagctctctatggtcgtcttttcctctggttggtcaaaaagatcaacaatgtcctctgtcaagagagaaaagcttactttattggtgttttggatatttcaggttttgaaattttcaaagtcaattcattcgaacaattatgtatcaattataccaatgaaaaactccaacaattcttcaatcaccatatgttcaaattggaacaagaagaatatcttaaagagaaaatcaattggactttcatcgattttggtcttgattcacaagccactatcgatttaattgatggtcgtcaaccaccaggtattttagctcttttggatgaacaatctgttttcccaaatgccaccgataatactttaatcaccaaactccacagtcactttagcaagaagaacgccaaatacgaagaaccacgtttctccaaaaccgaatttggtgttacccattatgctggtcaagtcatgtatgagattcaagattggttagaaaagaacaaagatccattacaacaagatctcgaactttgcttcaaagattcatcagacaacgttgtcaccaaacttttcaatgatccaaacattgccagtcgtgcaaagaaaggtgcaaactttatcactgtcgccgctcaatacaaggaacaattagcctcactcatggcnacccttgaaaccaccaacccacatttcgttcgttgtatcattccaaacaacaaacaattaccagccaaactcgaagataaagttgtcctcgaccaattacgttgcaatggtgtcctcgaaggtattcgtattactcgtaaaggtttcccaaatcgtattatctatgccgatttcgtcaaacgttactatttattagctccaaacgttccaagagacgctgaagactcacaaaaagccaccgatgctgttctcaaacatcttaacattgatccagaacaatatcgtttcggtatcaccaagattttcttccgtgccggtcaattagctcgtattgaagaagctcgtgaacaacgtctaggatccgaacaaaccaaatctgattatcttaaaagagccaatgaactcgttcaatggattaacgataaacaagcatcacttgaatcacgtgattttggtgattccatcgaatctgttcaaagtttcatgaacgctcataaagaatataaaaaaaccgaaaaaccaccaaagggtcaagaagtctctgaattggaagctatctacaattcattacaaactaaattacgtttaattaaacgtgaaccatttgttgcaccagctggtctcactccaaatgaaatcgattccacttggtccgctttagagaaagctgaacaagaacatgctgaagccctccgtattgaactcaaacgtcaaaagaaaattgcagttctcttacaaaaatacaatcgtattctcaagaaactcgaaaactgggccaccaccaaatctgtctacctcggttccaatgaaaccggtgacagtatcactgctgttcaagctaaattaaagaatttagaagcttttgatggtgaatgtcaatcattggaaggtcaatcaaactctgatctcctcagcattcttgctcaattaactgaactcaactacaatggtgtaccagaactcactgaacgtaaagatacattctttgctcaacaatggactggtgttaaatcatctgctgaaacctacaaaaacactcttttagctgaacttgaaagactccaaaagattgaagatgcattacatcatcatcatcatcatcaccac